(19) United States
(12) Patent Application Publication
(10) Pub. No.: US 2012/0071674 A1
(43) Pub. Date: Mar. 22, 2012

(54) SOLVATES OF DOCETAXEL

(75) Inventor: Witold Stanislaw Cieslinski, Lodz (PL)

(73) Assignee: PRZEDSIEBIORSTWO PRODUKCYJNO-WDROZENIOWE IFOTAM SP. Z O.O., LODZ (PL)

(21) Appl. No.: 13/322,643

(22) PCT Filed: May 28, 2010
(86) PCT No.: PCT/PL2010/000041

§ 371 Date: Nov. 28, 2011

(30) Foreign Application Priority Data
May 29, 2009 (PL) P-388144

(51) Int. Cl. C07D 305/14

(52) U.S. Cl. 549/510

(57) ABSTRACT

The present invention provides solvates of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C2-3-alkyl esters of formic acid, process of their preparation and their use in the synthesis of the pharmaceutically pure 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate.

### THE FIELD OF INVENTION

The present invention relates to new solvates of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and process for their preparation. The invention also concerns the use of new solvates in the process for preparation of pharmaceutically pure 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate.

4-Acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate is an antineoplastic agent known under the International Nonproprietary Name (INN) of docetaxel. Docetaxel is represented by the molecular structure given below.

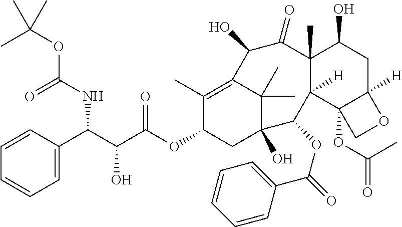

### BACKGROUND OF INVENTION

The prior art methods of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate preparation consist of esterification of selectively protected 10-deacetylbacatine III using the precursor of (2R,3S)-3-phenylisoserine and subsequent removal of groups protecting hydroxyl functions in positions C7, C10, and C2′ of the esterification product.

There are several approaches to achieve this goal in practice.

One of them is disclosed in EP 253738 B1, wherein the protected 10-deacetylbacatine III is esterified in the reaction with cinnamic acid or cinnamoyl chloride, followed by the reaction of the obtained intermediate with the sodium salt of tert-butyl N-chlorocarbamate and, optionally, removal of hydroxyl protecting groups.

The other known precursors of the side chain in the esterification process are, among others, linear derivatives of 3-tert-butoxycarbonylamino-2-hydroxy-3-phenylpropanoic acid (see, EP 336841 B1, WO 93/16059, WO 94/07876, WO 97/34866) or cyclic derivatives thereof, as oxazolidine derivatives (see, WO 92/09589, WO 94/07877, WO 94/07878, WO 94/07879, WO 94/10169, EP 735036 B1, WO 97/24345, WO 97/42167, U.S. Pat. No. 5,907,042, WO 02/12216), β-lactams (see, EP 617018 B1, WO 94/21623, U.S. Pat. No. 5,466,834, WO 2004/033442, WO 2006/135692), oxazinone derivatives (U.S. Pat. No. 5,254,703), aziridines (WO 2005/082875).

Protection of the hydroxyl groups in position C7 and C10 in 10-deacetylbacatine III and in position C2′ in the side chain is accomplished by the use of protecting groups well known in the art, eg. aryl, methoxymethyl, benzyloxymethyl, trialkylsilyl, (β-trimethoxysilylethoxy)methyl, tetrahydropyranyl, 2,2,2-trichloroathoxycarbonyl, 1-ethoxyethyl, benzyloxycarbonyl, chloroacetyl, imidazolecarbonyl, benzyl, 2,2,2-trichloroethyl, 2-(2-trichloromethylpropyl), 2,4-dinitrophenylsulfonyl and others. The protecting groups are removed under acidic or basic conditions by means of eg. zinc (for the removal of trichloroethoxycarbonyl) or catalytical hydrogenolysis (in the case of benzyloxycarbonyl).

Due to the low reactivity of C13-OH group in 10-deacetylbacatine III caused by steric hindrance, the esterification reaction is typically carried out for a long time at high temperatures, and using a large excess of acylating agent, resulting in low yield of the product. Moreover, epimerization of C2′-OH hydroxyl group in the phenylisoserine residue leads to the formation of several degradation products, which are difficult to remove from the reaction product.

4-Acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate trihydrate is the active pharmaceutical ingredient of the injection formulation Taxotere®. The crystalline structure as well as X-ray powder diffraction pattern (XRPD) of docetaxel trihydrate are described in J. Phys. IV France 11 (2001) Pr10-221. Examination of dehydration process of trihydrate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate proved also the existence of crystalline hemihydrates of docetaxel, further characterized by X-ray powder diffraction.

WO 96/01815 discloses 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate trihydrate, which is prepared by crystallization from the mixture of water and aliphatic alcohol, eg. water and ethanol, in the presence, or without, of ascorbic acid.

Further studies presented in Materials Science Forum vols. 443-444 (2004) pp 411-0 characterize crystalline structures of two polymorphic forms of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate trihydrate, namely form A and form B.

The prior art methods of purification of docetaxel, which is especially valuable substance because of limited sources of natural 10-deacetylbacatine III, are labour- and time-consuming, and consequently substantially insufficient.

EP 0253738 B1 discloses the process of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate purification by thin layer liquid chromatography using methylene chloride-methanol 97:3 mixture as eluent.

EP 0336841 B1 describes the preparation of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate with 90% purity by chromatography on silica gel Geduran using hexane/ethyl acetate 1:1 as eluent.

According to WO 93/06079, purification of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate on chromatographic column filled with silica gel yields product with 95% purity.

In WO 94/21622, 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate is purified by centrifugal partition chromatography (CPC) yielding product with 99.7% purity and 80.5% theoretical yield.

In WO 96/01815, 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate trihydrate having 98.7% purity is obtained as a result of crystallization of the starting material with 92.4% purity from ethanol and water.

U.S. Pat. No. 6,881,852 B2 describes four step process of purification starting from 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate of 70% purity comprising refinement in the mixture methanol-water, crystallization from the mixture acetone-hexane, followed by crystallization from the mixture methanol-water, and finally crystallization from the mixture acetone-hexane. Said process yields product with the final purity of 99.65% and 42% overall yield.

Similar process of purification is described in U.S. Pat. No. 6,838,569 B1. The purification comprises refinement in the mixture methylene chloride-hexane, two-fold crystallization from the mixture acetone-hexane and one more crystallization from the mixture acetonitrile-water. The process results in docetaxel trihydrate of 99.59% purity with 39% overall yield.

According to U.S. Pat. No. 6,900,342 B1,4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate is purified by column chromatography on silicagel 60 using ethyl acetate-hexane 6:4 as eluent.

In WO 2006/037653, crude 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate, after isolation from reaction mixture by crystallization from ethanol and water, is subsequently purified by two crystallizations, first from ethanol-water and then from acetone-heptane. This process allows to obtain product of 99.4% purity with 83% yield.

In WO 2006/004896, crude 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate is purified by column chromatography with the use of solvents system methylene chloride-ethyl acetate or acetone as eluent.

WO 2007/109654 discloses two methods for 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate purification, first by column chromatography on silicagel with the use of the mixture ethyl acetate-n-heptane, 4:1 and 1:1 as eluent, and the second by refinement in acetone, with 67% yield of the product and more than 99% purity.

The method of purification of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate described in WO 2008/051465 comprises filtration of its solution in ethyl acetate through Celite®, activated, charcoal and active acidic bad, followed by column chromatography with the use of methylene chloride.

According to US 20080200699, the crude 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate is purified by chromatography using methylene chloride/methanol 95:5 as eluent.

U.S. Pat. No. 7,332,617 B2 describes process for preparation of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate trihydrate which comprises dissolving anhydrous docetaxel of initial chromatographic purity above 99.5% in acetone, concentrating of the reaction mixture, repeated dissolving of thus obtained oily substance in acetone and finally crystallization from water-acetone mixture.

The aforementioned methods of purification of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate are either chromatographic methods requiring the use of the large amounts of solvents which prolongs the process and increases its costs or the methods consisting in multiple refinements or crystallizations. Even though they are simpler and do not need the large amounts of solvents, the obtained products have low overall yields.

In view of the standards set by International Conference on Harmonisation of Technical Requirements for Registration of Pharmaceuticals for Human Use (ICH), the pharmaceutically active ingredients have to comply with appropriate purity requirements, ie. to characterize by so called pharmaceutical purity. Thus, the development of the effective process for preparation of high pharmaceutical purity docetaxel is still a problem of great importance.

### SUMMARY OF THE INVENTION

The aforementioned drawbacks of the processes for the preparation of pharmaceutically pure 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate can be avoided by implementing into the process the purification of the crude product isolated from post-reaction mixture in anhydrous or hydrate forms, e.g. in trihydrate form. The said step of purification comprises the conversion of the crude product into its crystalline solvate with C₂₋₃-alkyl ester of formic acid.

Whenever a reference is made hereafter to “pharmaceutically pure” 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate, it is to be understood that it is 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate in anhydrous or hydrate forms, e.g. in any polymorphic trihydrate form, comprising less than 0.1% of a single unidentified impurity or less than 0.4% of unidentified impurities in total.

The first aspect of the present invention provides the new solvates of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃-alkyl esters of formic acid.

The solvates according to the invention are selected from the group of solvates of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate with ethyl ester of formic acid, propyl ester of formic acid, and isopropyl ester of formic acid.

The conversion of the crude synthesis product into its crystalline solvate with C₂₋₃-alkyl ester of formic acid according to the present invention makes the process of preparation of pharmaceutical purity 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate much simpler and more economical. A single or double crystallization allows to obtain the solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate with high yield of the step (above 90%) and of chromatographic purity (by HPLC) higher than 99%, without any need to use chromatographic methods of purification and multiple refinements or crystallizations.

The starting material, crude 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate, can be in any form and any degree of purity, eg. purity not higher than 95-98%.

The second aspect of the present invention is the process for preparation of the solvates of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃-alkyl esters of formic acid comprising crystallization of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate, in either anhydrous or hydrate forms, especially trihydrate form, from the mixture of methylene chloride and C₂₋₃-alkyl ester of formic acid, followed by drying of the obtained solvate to remove free solvents.

Another aspect of the invention is the process of preparation of pharmaceutically pure 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate, in either anhydrous or hydrate forms, especially trihydrate form, via the conversion of crude 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate into its crystalline solvate with C₂₋₃-alkyl ester of formic acid and subsequent desolvation thereof.

### BRIEF DESCRIPTION OF FIGURES

FIG. 1. Thermogravimetric analysis (TGA) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid.

FIG. 2. Thermogravimetric analysis (TGA) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid.

FIG. 3. Thermogravimetric analysis (TGA) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid.

FIG. 4. Thermogram of differential scanning calorimetry (DSC) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid.

FIG. 5. Thermogram of differential scanning calorimetry (DSC) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid.

FIG. 6. Thermogram of differential scanning calorimetry (DSC) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid.

FIG. 7. FT-IR spectrum (Nujol) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid.

FIG. 8. FT-IR spectrum (Nujol) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid.

FIG. 9. FT-IR spectrum (Nujol) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid.

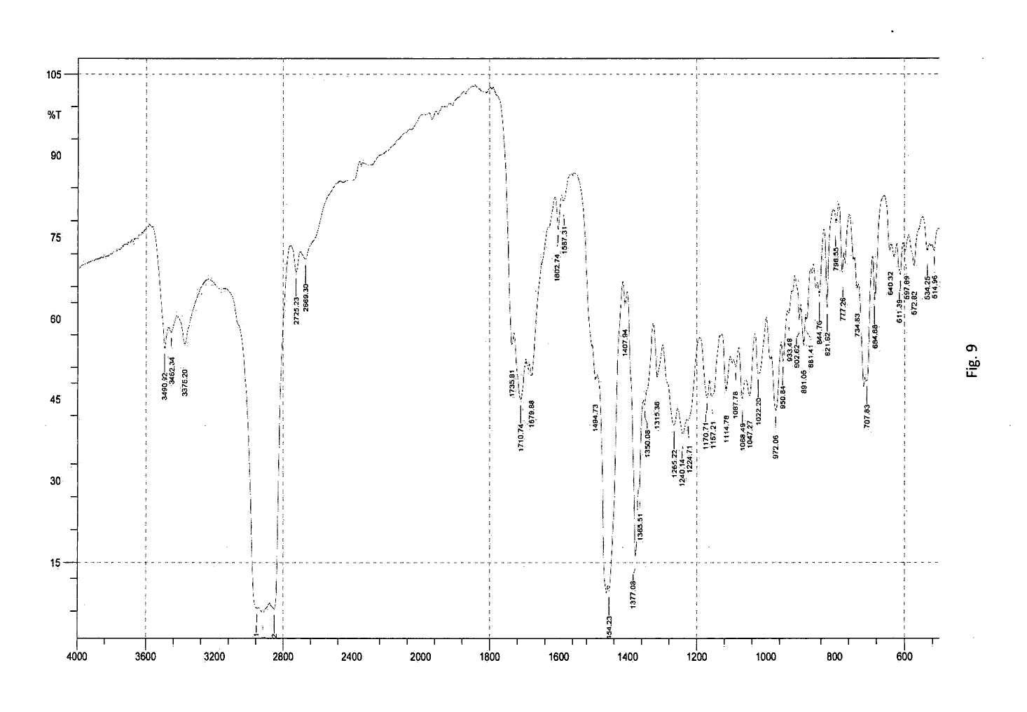

FIG. 10. X-ray powder diffraction pattern (XRPD) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid obtained according to Example 1.

FIG. 11. X-ray powder diffraction pattern (XRPD) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid obtained according to Example 2.

FIG. 12. X-ray powder diffraction pattern (XRPD) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid obtained according to Example 3.

FIG. 13. Microscopy image of single crystal of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid (optical microscope Meiji Techno MX4310H).

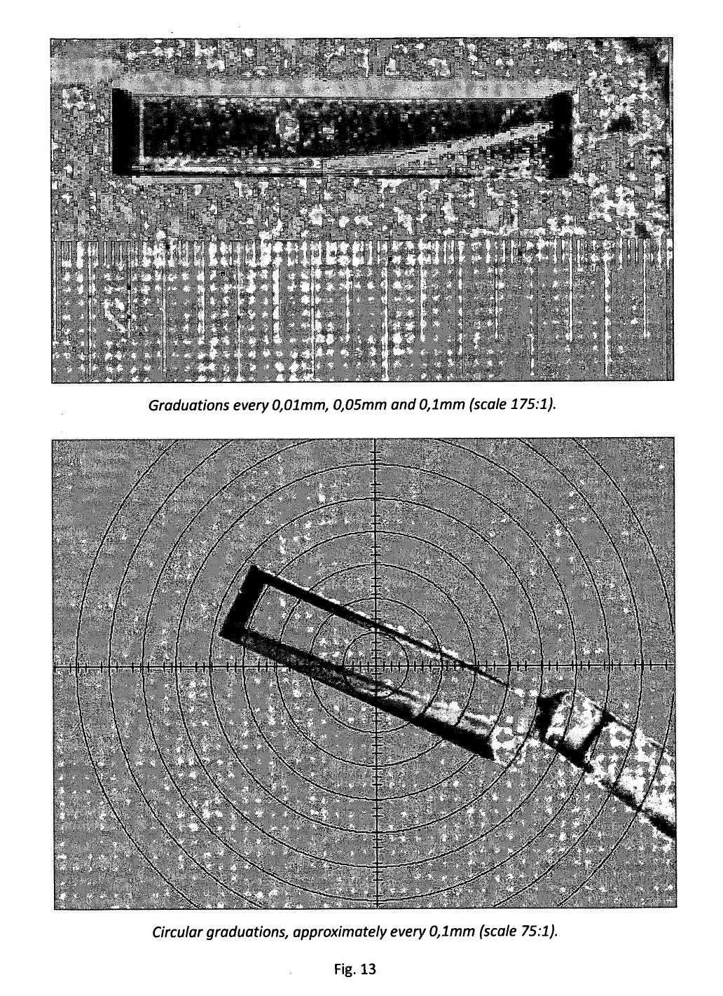

FIG. 14. Crystalline structure of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid.

FIG. 15. X-ray powder diffraction pattern (XRPD) of single crystal of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid.

FIG. 16. Computer simulated X-ray powder diffraction pattern (XRPD) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid obtained from single crystal X-ray diffraction studies.

FIG. 17. Computer simulated X-ray powder diffraction pattern (XRPD) obtained from single crystal X-ray diffraction studies (lower line) and experimentally obtained X-ray powder diffraction pattern (XRPD) obtained from single crystal x-ray diffraction studies (upper line) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid, range of 2theta from 3 to 30 degrees.

FIG. 18. Hydrogen bonds in solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid:

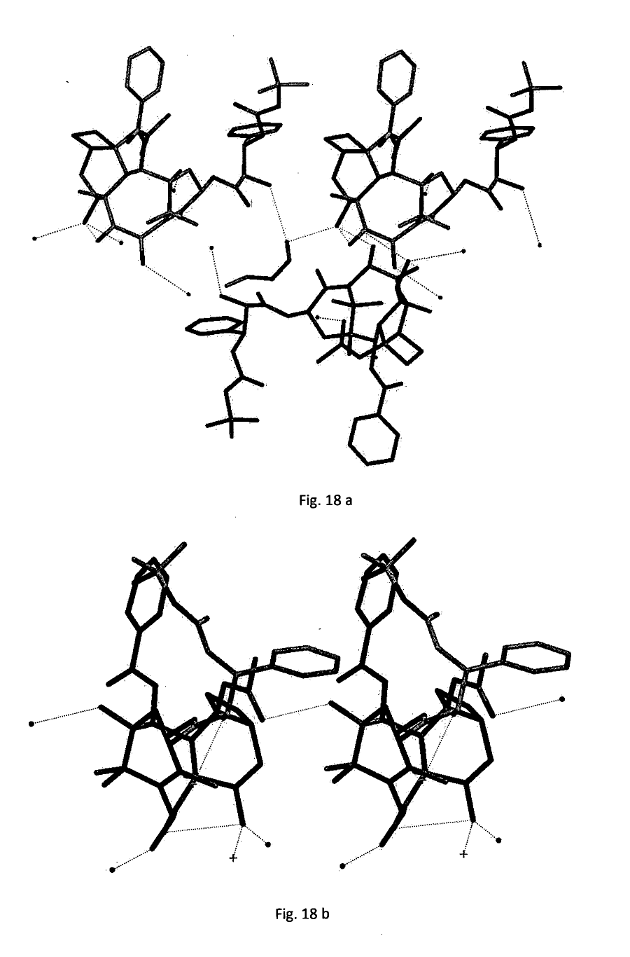

a) hydrogen bonds HO-2′-C═O(HCOOEt)-HO-7-HO-10 and C₉═O,

b) hydrogen bonds C═O(Ac-4)-HO-1.

FIG. 19. Crystalline structure of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid obtained from single crystal x-ray diffraction studies.

FIG. 20. Computer simulated X-ray powder diffraction pattern (XRPD) obtained from single crystal x-ray diffraction studies of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid.

FIG. 21. Hydrogen bonds in solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid:

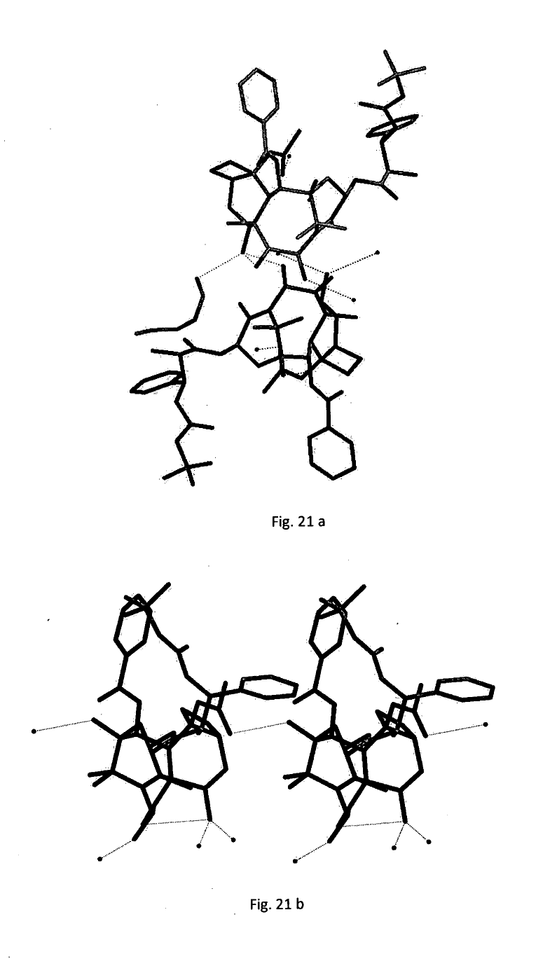

a) hydrogen bonds C═O(HCOOPO—HO-7-HO-10 and C₉═O,

b) hydrogen bonds C═O(Ac-4)-HO-1.

FIG. 22. Crystalline structure of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid obtained from single crystal x-ray diffraction studies.

FIG. 23. Computer simulated X-ray powder diffraction pattern (XRPD) obtained from single crystal x-ray diffraction studies of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid.

FIG. 24. Hydrogen bonds in solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid:

a) hydrogen bonds C═O(HCOOiPr)—HO-7-HO-10 and C₉═O,

b) hydrogen bonds C═O(Ac-4)-HO-1.

### DETAILED DESCRIPTION OF THE INVENTION

The solvates of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃-alkyl esters of formic acid according to the present invention are obtained in the process comprising:
a) dissolving of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate in methylene chloride,
b) evaporation of methylene chloride,
c) addition of C₂₋₃ alkyl ester of formic acid,
d) optionally, seeding the crystallization mixture with crystals of the solvate,
e) leaving the crystallization mixture with, or without, stirring to form the crystals of the solvate,
f) isolation of the formed crystals,
g) washing of the crystals with C₂₋₃ alkyl ester of formic acid,
h) drying of crystals to constant weight.

The process according to the invention is preferably carried out in the following way. The starting material crude 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate, being the product of the synthesis, optionally can be pre-purified to the level of chemical purity ca. 90-95% (determined by HPLC). The crude 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate can be either anhydrous or in hydrate form, especially trihydrate form.

That starting material is dissolved in excess methylene chloride, preferably at room temperature. In the preferred embodiment of the invention, the dissolving process is realized on Schott type funnel with simultaneous filtering off solid impurities or filtration is carrying on after dissolving. The amount of methylene chloride necessary for dissolving is up to 22 L per 1 kg of starting material.

After insoluble impurities removal, distillation of methylene chloride is carried on. Generally, the distillation of methylene chloride is accomplished under reduced pressure with bath temperature ca. 40° C., and the internal temperature of the reaction mixture 25±5° C. The process is continued until reaching the concentration of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate higher than 120 g/L.

Then, to the reaction mixture C₂₋₃-alkyl ester of formic acid is added in the amount of about 880 mL per 1 kg of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and the removal of solvents is continued until reaching concentration higher than 160 g per 1 L.

In the preferred embodiment of the invention, removal of methylene chloride is accomplished with simultaneous addition of C₂₋₃-alkyl ester of formic acid.

Stirring of the reaction mixture is continued at room temperature (heating bath is removed) till solvate crystals starts forming. Optionally, the crystallizing mixture can be seeded with the crystals of solvate. Typically, the crystals form within one hour. The mixture is left then for further crystallization with, or without, stirring. After solvate crystals formation, crystallizing mixture is cooled to 0-3° C. and is left without stirring.

The formed crystals are filtered and washed, preferably three times, with C₂₋₃-alkyl ester of formic acid chilled to temperature below 5° C.

Then, the crystals of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃-alkyl ester of formic acid are dried, preferably in the stream of air of room temperature for about one hour, until constant weight is reached.

The structures of the obtained solvates of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃-alkyl esters of formic acid were conformed by the methods of thermogravimetric analysis (TGA), differential scanning calorimetry (DSC), proton nuclear magnetic resonance spectroscopy (¹H NMR), Fourier transform infrared spectroscopy (FT-IR), X-ray powder diffraction (XRPD) and X-ray single crystal studies.

### Characteristic of Solvates

### Solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and Ethyl Ester of Formic Acid

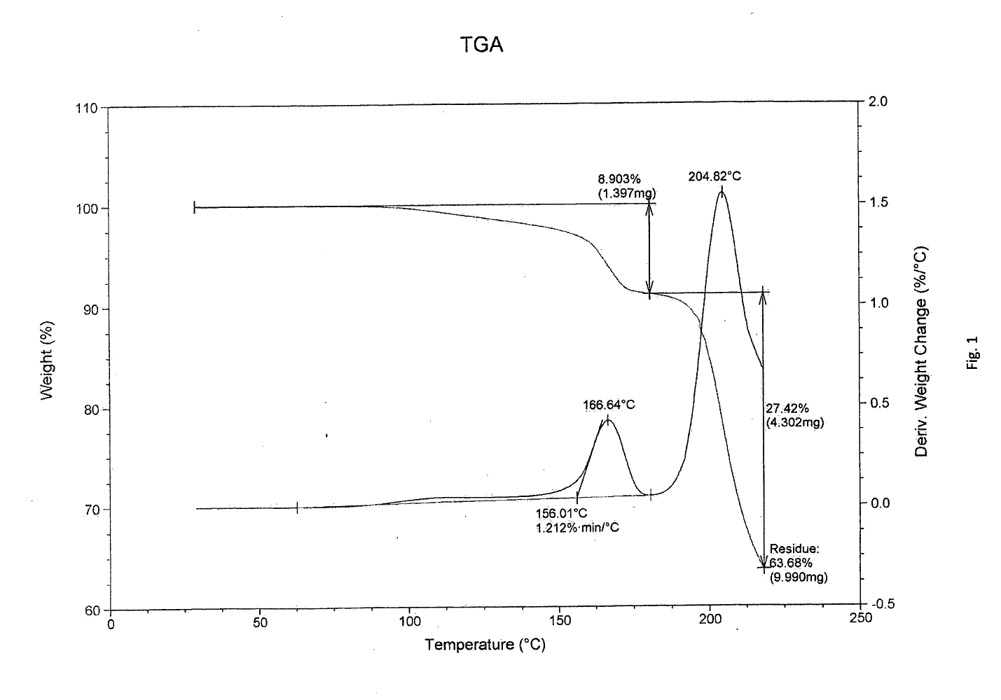

TGA curve (FIG. 1) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid shows approximately 8.903% weight loss in the temperature range 30-180° C., especially in range 80-174° C. After deduction of 0.206% of weight, which correspond to free water determined by Karl-Fisher titration, the real weight loss corresponds to the content of ethyl ester of formic acid in solvate 8.697% of initial sample weight. Theoretical content of ethyl ester of formic acid in monosolvate is 8.40%.

¹H NMR spectra of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid conforms stoichiometric composition of the solvate.

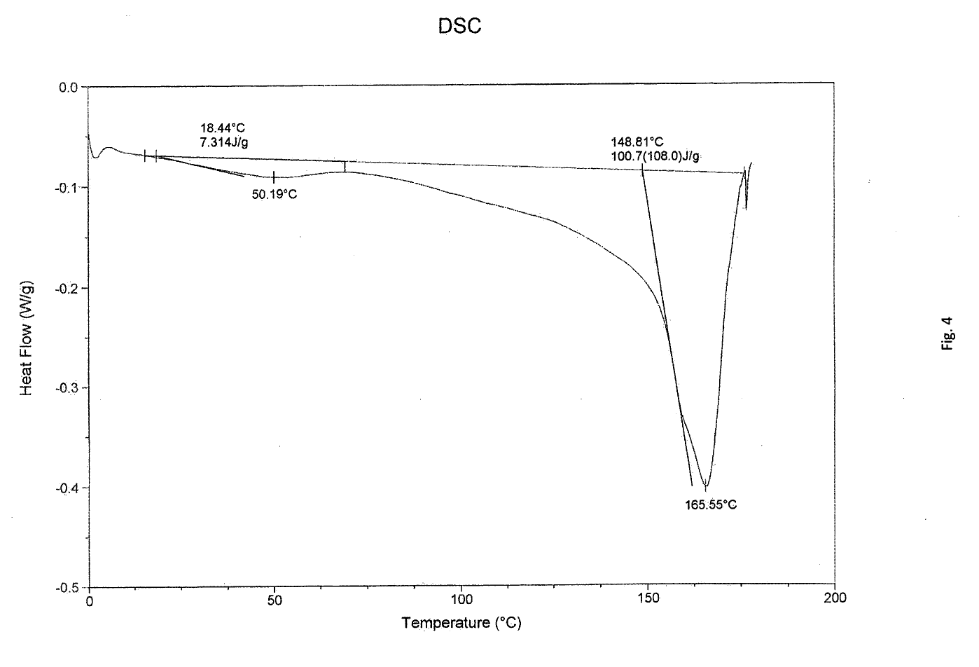

DSC (FIG. 4) of solvate 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid shows no presence of hydration water. Moreover, the peak corresponding to hydrate dissociation is not observed.

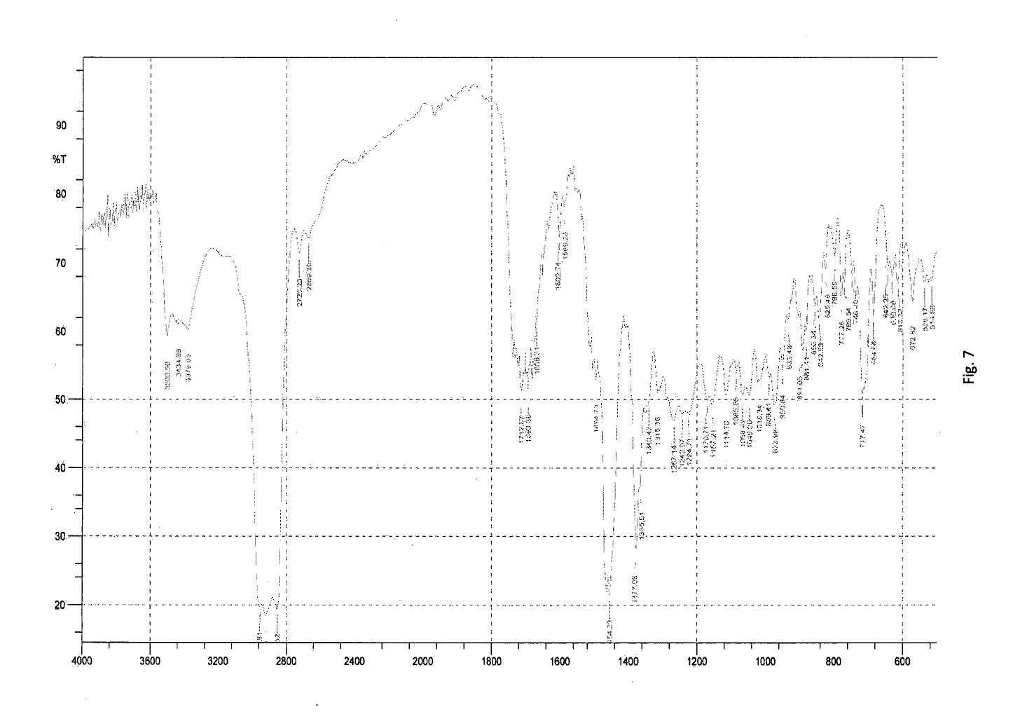

FT-IR spectrum (Nujol) (FIG. 7) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid is characterized by bands at: 974, 1049, 1068, 1115, 1157, 1171, 1225, 1242 and 1267 cm⁻¹. The spectrum is characterized by additional bands at 1713, 3379, 3435 and 3501 cm⁻¹.

The solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid is characterized by X-ray powder diffraction (XRPD) pattern having peaks at 2θ values at: 4.5°; 7.1°; 8.8°; 11.2°; 14.1°; 17.4° and 18.5°±0.2°. The solvate can be characterized in detail by additional peaks at 2θ values at: 12.2°; 13.6°; 14.6°; 15.4°; 16.7°; 20.5°; 22.0° and 24.4±0.2°.

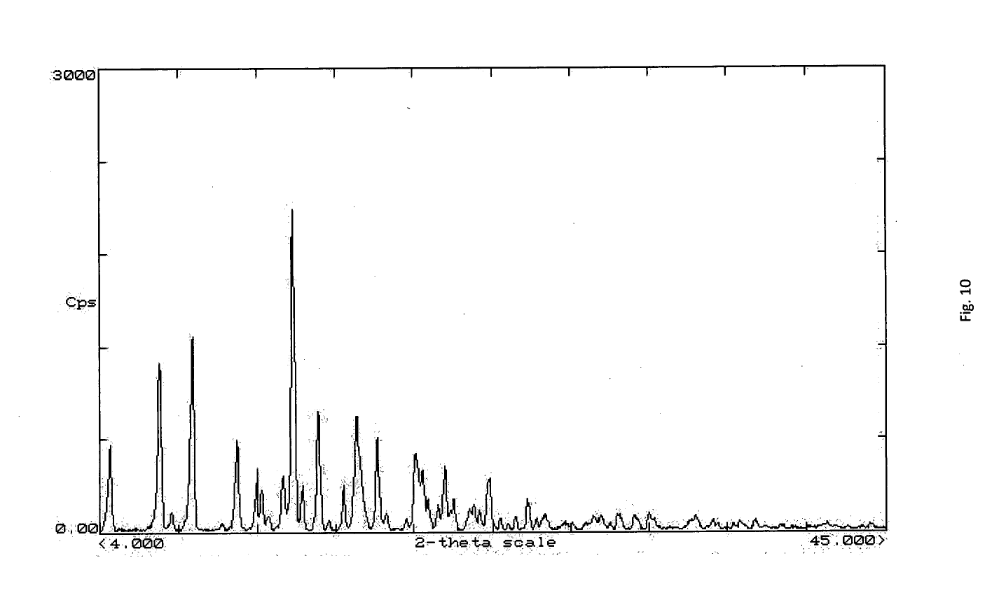

XRPD pattern of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid acquired with CuKα₁ radiation source with wave length λ=1.54056 Å as the relation of relative intensity (I/Iₒ) dependence on 2θ is substantially similar to the one shown in FIG. 10.

In Table 1 are shown the 2θ values, interplanar distances d and relative intensities I/Iₒ (relative intensities higher than 10%) in XRPD pattern of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid.

TABLE 1
XRPD of solvate of 4-acetoxy-2α-benzoyloxy-5β,20-epoxy-1,7β,10β-
trihydroxy-9-oxo-tax-11-en-13α-yl (2R,3S)-3-tert-butoxycarbonylamino-
2-hydroxy-3-phenylpropionate and ethyl ester of formic acid

| 2θ [°] | d [Å] | I/Iₒ [%] |
|---|---|---|
| 4.48 | 19.71 | 26 |
| 7.08 | 12.48 | 52 |
| 8.84 | 10.00 | 60 |
| 11.16 | 7.92 | 28 |
| 12.20 | 7.25 | 19 |
| 12.44 | 7.11 | 13 |
| 13.56 | 6.53 | 17 |
| 14.08 | 6.29 | 100 |
| 14.56 | 6.08 | 14 |
| 15.40 | 5.75 | 37 |
| 16.72 | 5.30 | 14 |
| 17.40 | 5.09 | 35 |
| 18.52 | 4.79 | 29 |
| 20.52 | 4.33 | 24 |
| 20.88 | 4.25 | 18 |
| 22.04 | 4.03 | 20 |
| 24.36 | 3.65 | 16 |

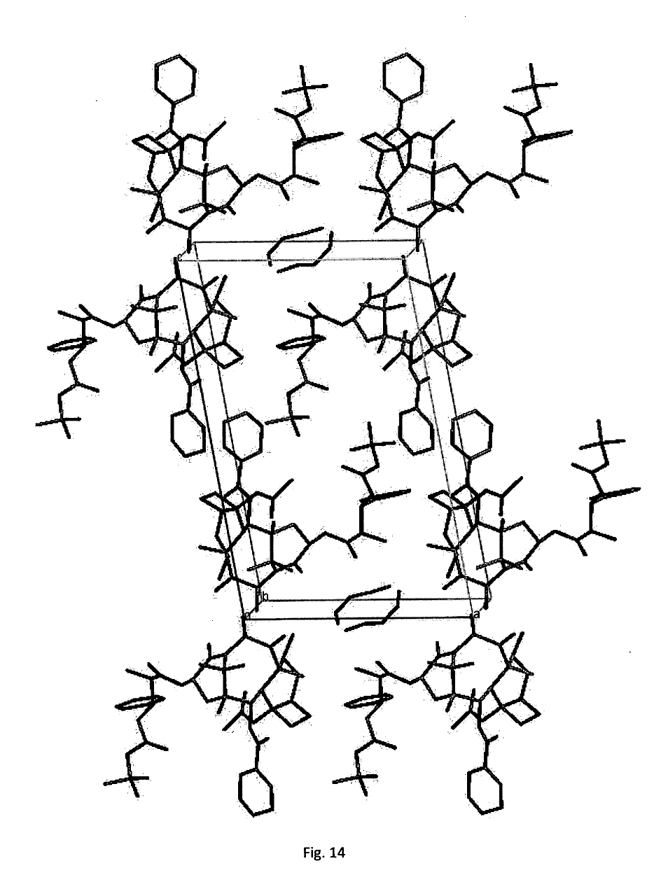

The crystalline structure of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid was determined from single crystal X-ray diffraction data (FIG. 14). The analysis conformed the presence of both 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid in the crystal lattice. Type of unit cell and parameter values of crystal lattice determined from single crystal X-ray diffraction conform that the crystal of solvate belongs to space group P2(1). Solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid crystallizes in monoclinic crystal system. Number of molecules in elemental cell Z is equal to 2, considering calculated density d equal to 1.295. The full characteristic of crystalline form as list of parameters of unit cell is presented in Table 2.

TABLE 2
Parameters of unit cell of solvate of 4-acetoxy-2α-benzoyloxy-5β,20-
epoxy-1,7β,10β-trihydroxy-9-oxo-tax-11-en-13α-yl (2R,3S)-3-tert-
butoxycarbonylamino-2-hydroxy-3-phenylpropionate and ethyl ester of
formic acid

| Crystal system | Monoclinic |
|---|---|
| Space group | P2(1) |
| a (Å) | 12.8333(5) |
| b (Å) | 8.7056(3) |
| c (Å) | 20.5241(8) |
| α (°) | 90 |
| β (°) | 99.344(1) |
| γ (°) | 90 |
| Z | 2 |
| V (Å³) | 2262.56 |
| D | 1.295 |
| R (%) | 4.69 |
| R_{w}(%) | 14.11 |
| G.o.F. | 1.105 |
| Reflections | 13545 |
| (Fₒ > 4sig(Fₒ)) | |
| Parameters refined | 585 |

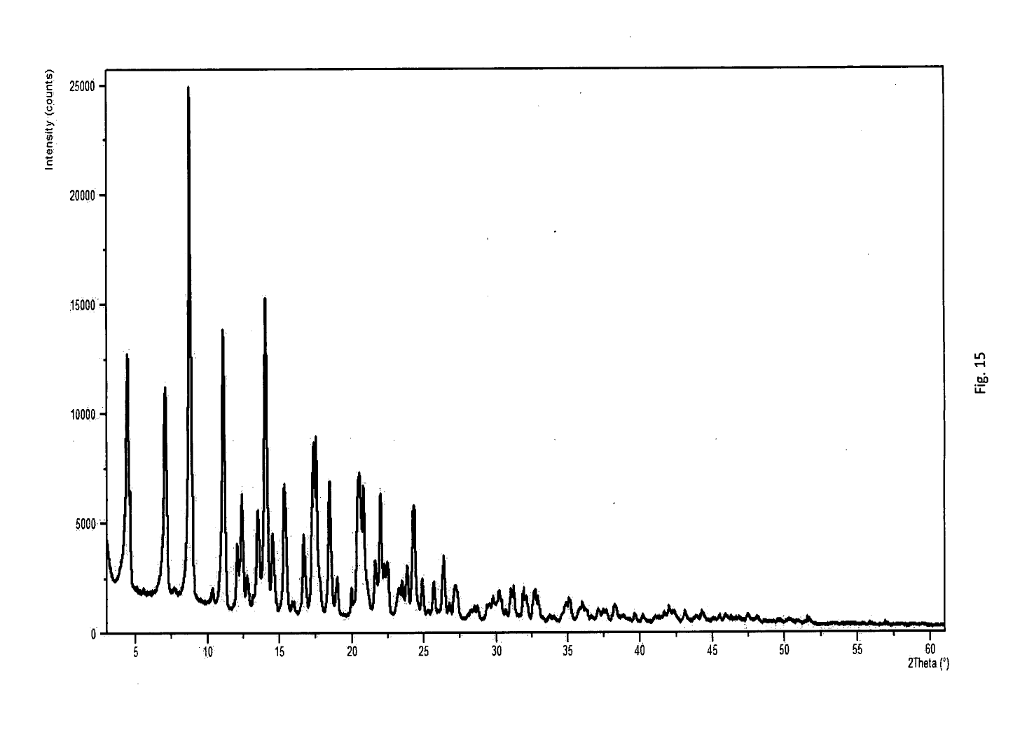

X-ray powder diffraction pattern of single crystal of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid acquired with CuKα₁ radiation source at wave length λ=1.541874 Å as the relation of 2θ values and interplanar distances d values for peaks with relative intensity (I/Iₒ) higher than 10%, are shown in FIG. 15 and in Table 3.

TABLE 3
XRPD of single crystal of solvate of 4-acetoxy-2α-benzoyloxy-5β,20-
epoxy-1,7β,10β-trihydroxy-9-oxo-tax-11-en-13α-yl (2R,3S)-3-tert-
butoxycarbonylamino-2-hydroxy-3-phenylpropionate and ethyl ester of
formic acid

| 2θ [°] | d [Å] | I/Iₒ [%] |
|---|---|---|
| 4.47 | 19.76 | 44.2 |
| 7.10 | 12.45 | 39.1 |
| 8.77 | 10.08 | 100.0 |
| 8.97 | 9.86 | 22.4 |
| 11.09 | 7.98 | 52.3 |
| 12.07 | 7.33 | 12.7 |
| 12.41 | 7.13 | 21.8 |
| 13.49 | 6.56 | 18.5 |
| 14.06 | 6.30 | 61.6 |
| 14.53 | 6.10 | 14.4 |
| 15.32 | 5.78 | 22.8 |
| 15.41 | 5.75 | 21.8 |
| 16.67 | 5.32 | 15.2 |
| 17.38 | 5.10 | 33.5 |
| 17.55 | 5.05 | 32.8 |
| 18.46 | 4.81 | 25.9 |
| 20.42 | 4.35 | 24.6 |
| 20.55 | 4.32 | 27.4 |
| 20.81 | 4.27 | 24.8 |
| 21.98 | 4.04 | 22.7 |
| 24.27 | 3.66 | 19.8 |
| 24.37 | 3.65 | 20.0 |
| 26.38 | 3.38 | 11.6 |

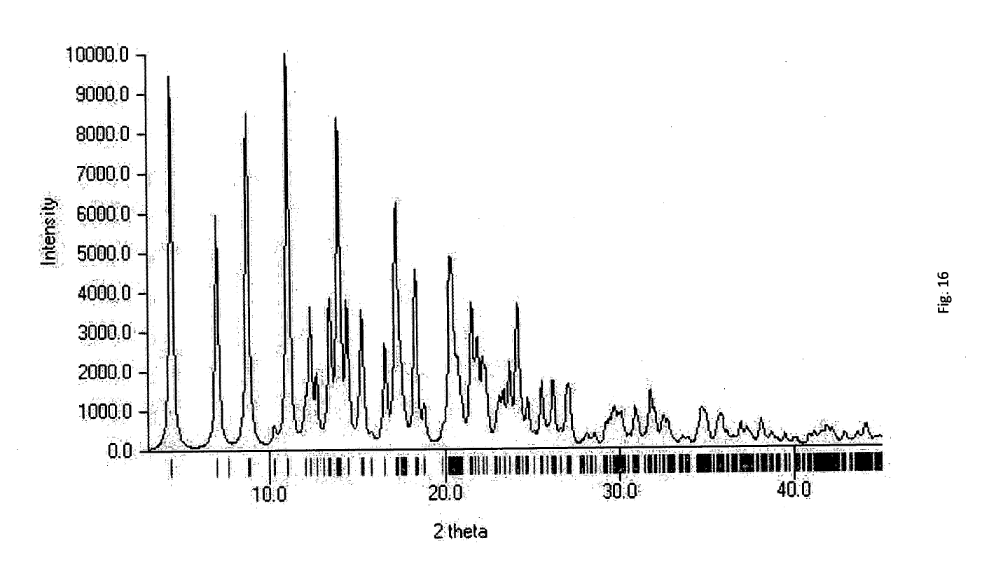

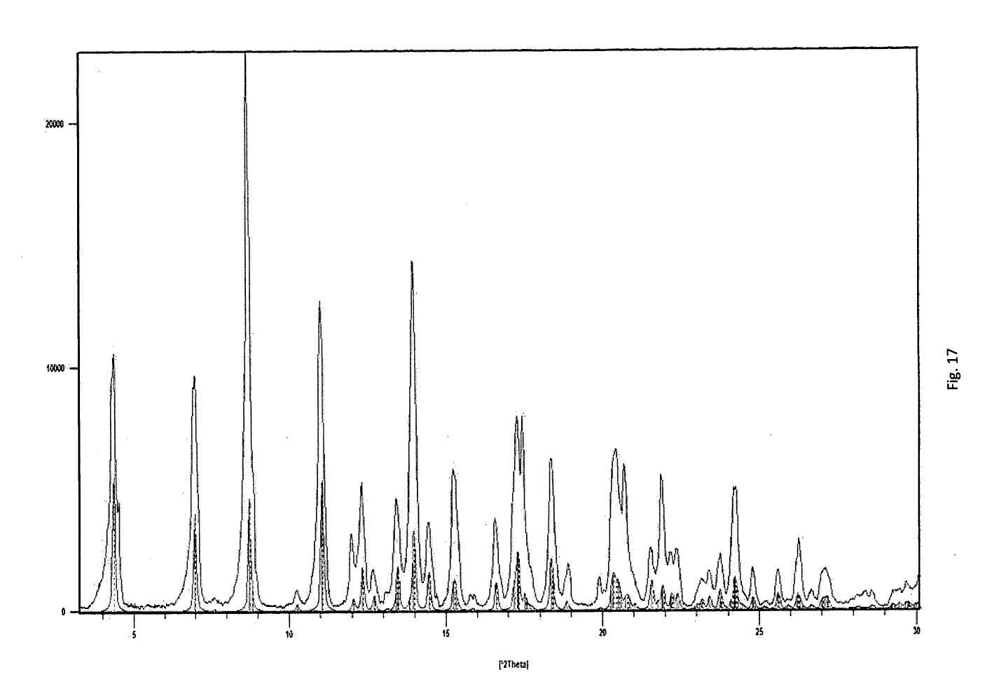

The experimental XRPD pattern of single monocrystal is analogous to the computer simulated pattern determined from single crystal X-ray diffraction data (FIG. 16). Theoretical (simulated) XRPD pattern determined from single crystal X-ray diffraction studies and experimental XRPD pattern overlapped completely in the range of 2θ values from 3 to 30 degrees (FIG. 17), conforming homogeneity of phase composition of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid. Based on X-ray studies of the single crystal of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid the hydrogen bonds in crystal were named. These bonds between the following groups of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid: HO-2′-C═O(HCOOEt)-HO-7-HO-10 and C₉═O and also between groups of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate: C═O(Ac-4)-HO-1.

### Solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and Propyl Ester of Formic Acid

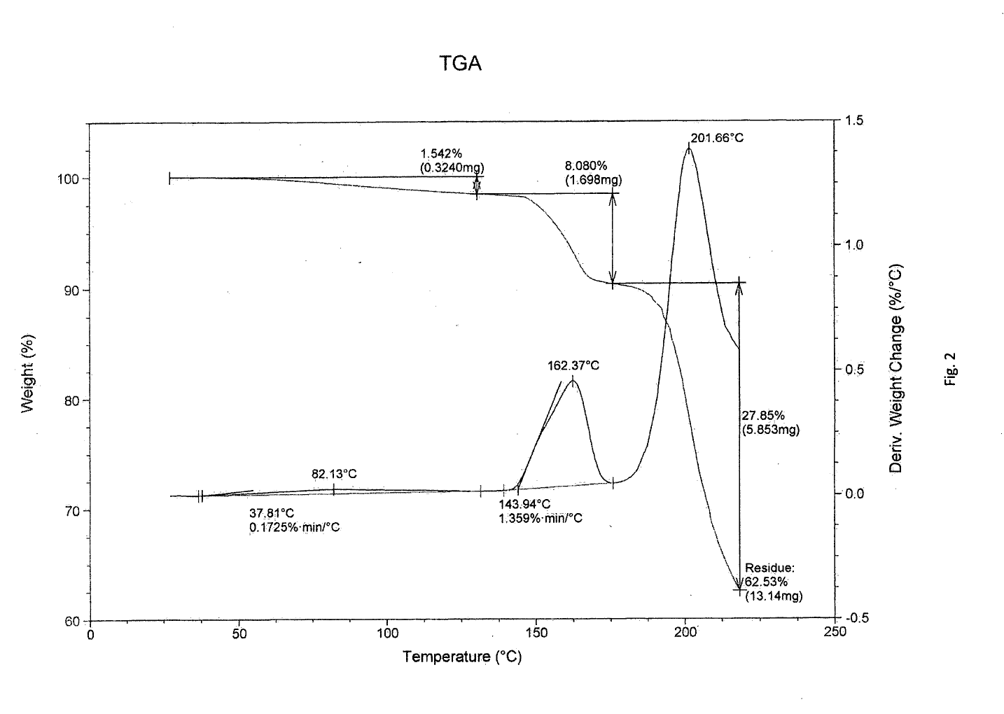

TGA curve (FIG. 2) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid shows 9.622% weight loss in the temperature range 30-180° C., especially in the range 55-175° C. After deduction of 0.181% of weight, which correspond to free water determined by Karl-Fisher titration, the real weight loss corresponds to the content of propyl ester of formic acid in solvate 9.441% of initial sample weight. Theoretical content of propyl ester of formic acid in monosolvate is 9.834%.

¹H NMR spectra of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid conforms stoichiometric composition of the solvate.

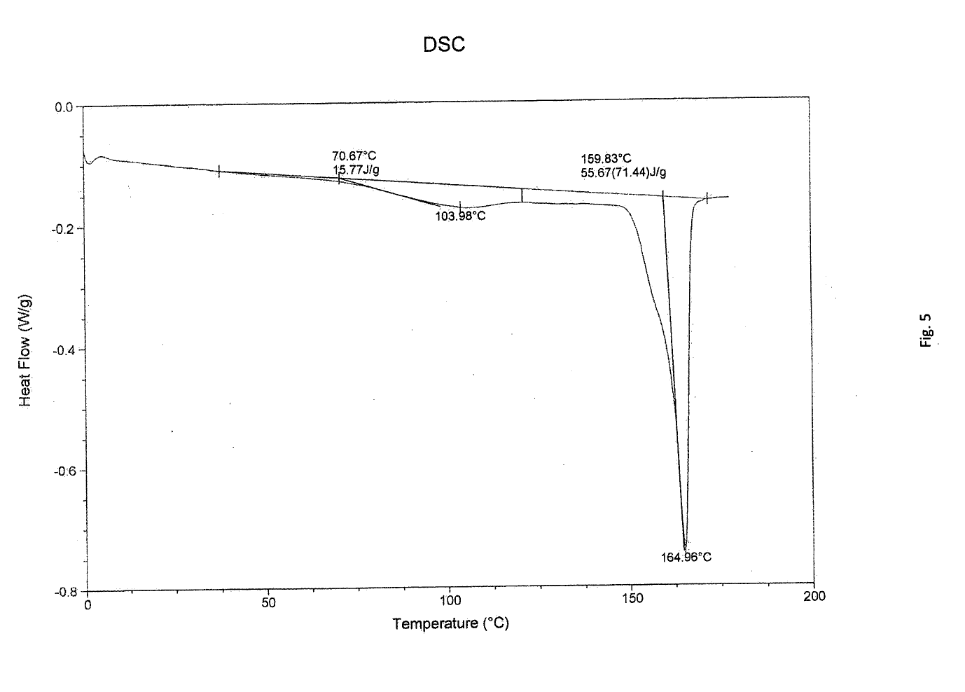

DSC (FIG. 5) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid shows no presence of hydration water.

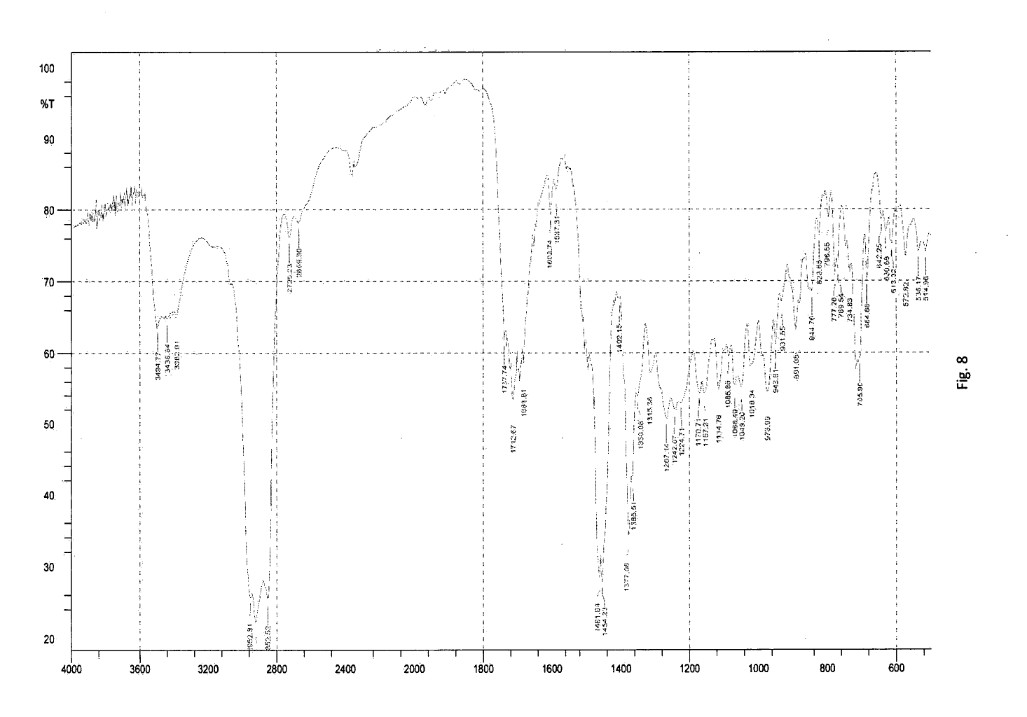

FT-IR spectrum (Nujol) (FIG. 8) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid is characterized by bands at: 974, 1049, 1068, 1115, 1157, 1171, 1225, 1242 and 1267 cm⁻¹. The spectrum is characterized by additional bands at 1713, 3383, 3439 and 3495 cm⁻¹.

The solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid is characterized by X-ray powder diffraction (XRPD) pattern having peaks at 2θ values at: 4.5; 7.1; 8.8; 11.1; 14.1; 15.4; 17.4; 18.5; and 20.4±0.2°. The solvate can be characterized in detail by additional peaks at: 12.1; 12.4; 12.8; 13.5; 14.6; 16.7; 18.9; 21.6; 22.0; 22.3; 22.4; 23.5; 23.8; 24.3; 24.8; 25.6; 26.3 and 27.1±0.2°.

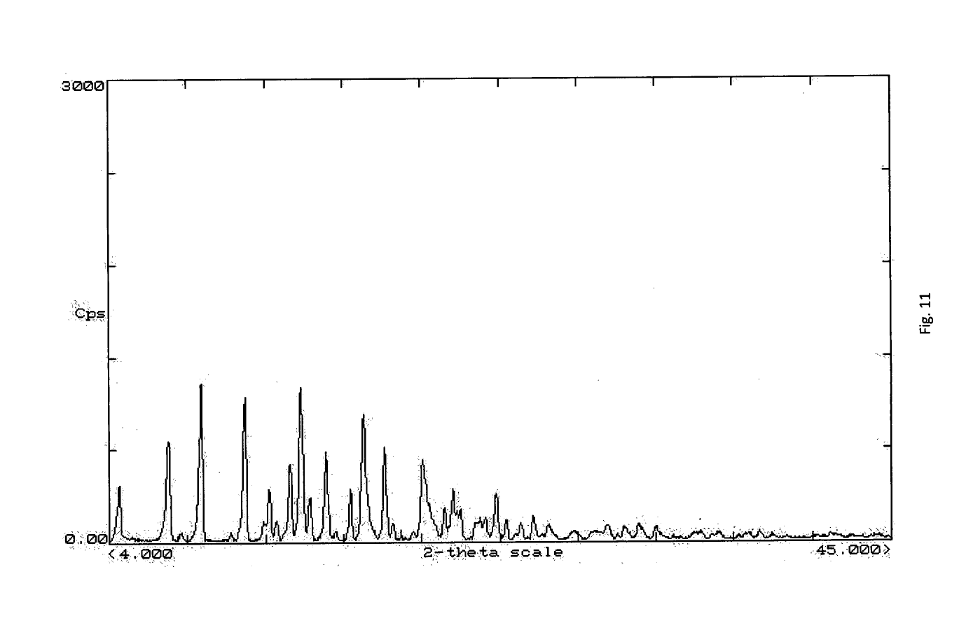

XRPD of solvate of 4-acetoxy-2α-benzoyloxy-5β,20-epoxy-1,7β,10β-trihydroxy-9-oxo-tax-11-en-13α-yl(2R,3S)-3-tert-butoxycarbonylamino-2-hydroxy-3-phenylpropionate and propyl ester of formic acid acquired with CuKα₁ radiation source at wave length λ=1.54056 Å as the relation of relative intensity (I/Iₒ) and 2θ is substantially similar to the one shown in FIG. 11.

In Table 4 are listed 2θ values, interplanar distances d values and relative intensities I/Iₒ (intensities higher than 15%) in XRPD of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid.

TABLE 4
XRPD of solvate of 4-acetoxy-2α-benzoyloxy-5β,20-epoxy-1,7β,10β-
trihydroxy-9-oxo-tax-11-en-13α-yl (2R,3S)-3-tert-butoxycarbonylamino-2-
hydroxy-3-phenylpropionate and propyl ester of formic acid

| 2θ [°] | d [Å] | I/Iₒ [%] |
|---|---|---|
| 4.48 | 19.71 | 45 |
| 7.08 | 12.48 | 69 |
| 7.76 | 11.38 | 17 |
| 8.84 | 10.00 | 100 |
| 10.40 | 8.50 | 16 |
| 11.12 | 7.95 | 92 |
| 12.12 | 7.30 | 23 |
| 12.40 | 7.13 | 41 |
| 12.80 | 6.91 | 23 |
| 13.52 | 6.54 | 55 |
| 14.08 | 6.29 | 100 |
| 14.56 | 6.08 | 39 |
| 15.40 | 5.75 | 65 |
| 15.88 | 5.58 | 22 |
| 16.68 | 5.31 | 46 |
| 17.36 | 5.10 | 88 |
| 18.48 | 4.80 | 70 |
| 18.88 | 4.70 | 28 |
| 19.32 | 4.59 | 24 |
| 19.52 | 4.53 | 20 |
| 19.96 | 4.45 | 23 |
| 20.44 | 4.34 | 63 |
| 21.60 | 4.11 | 35 |
| 22.04 | 4.03 | 46 |
| 22.28 | 3.99 | 33 |
| 22.44 | 3.96 | 34 |
| 23.48 | 3.79 | 28 |
| 23.76 | 3.74 | 29 |
| 24.32 | 3.66 | 41 |
| 24.84 | 3.58 | 26 |
| 25.32 | 3.52 | 17 |
| 25.64 | 3.47 | 23 |
| 26.28 | 3.39 | 26 |
| 27.08 | 3.29 | 21 |
| 30.16 | 2.96 | 18 |
| 31.08 | 2.88 | 17 |
| 31.80 | 2.81 | 18 |

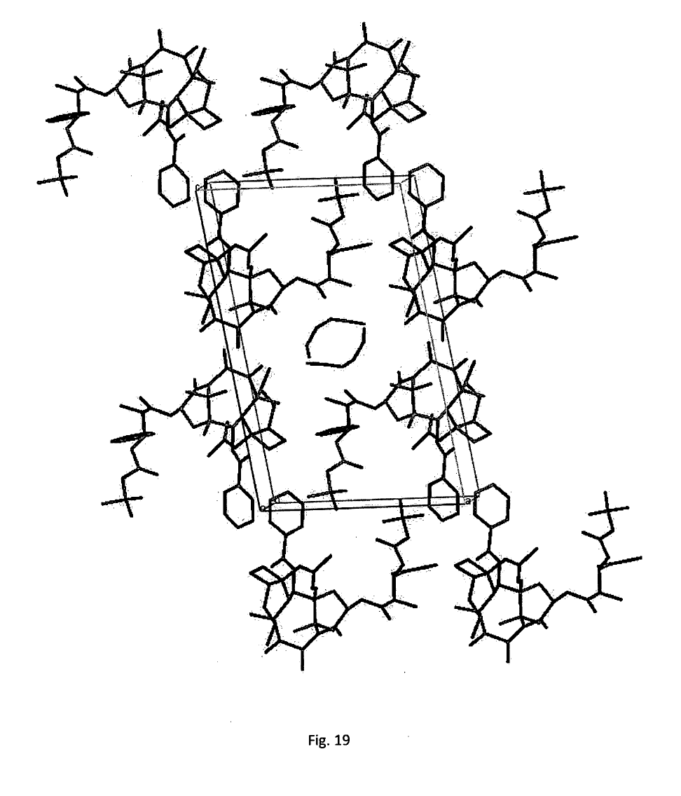

The crystalline structure (FIG. 19) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid was determined from single crystal X-ray diffraction data. The analysis conformed the presence of both 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid in the crystal lattice. Type of unit cell and parameter values of crystal lattice determined from single crystal X-ray diffraction conform that the crystal of solvate belongs to space group P2(1). Solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid crystallizes in monoclinic crystal system. Number of molecules in unit cell Z is equal to 2 considering calculated density d equal to 1.305. The full characteristic of crystalline form as list of parameters of unit cell is presented in Table 5.

TABLE 5
Parameters of unit cell of solvate of 4-acetoxy-2α-benzoyloxy-5β,20-
epoxy-1,7β,10β-trihydroxy-9-oxo-tax-11-en-13α-yl (2R,3S)-3-tert-
butoxycarbonylamino-2-hydroxy-3-phenylpropionate and propyl ester of
formic acid

| Crystal system | Monoclinic |
|---|---|
| Space group | P2(1) |
| a (Å) | 12.8499(11) |
| b (Å) | 8.7500(7) |
| c (Å) | 20.5396(17) |
| α (°) | 90 |
| β (°) | 99.052(1) |
| γ (°) | 90 |
| Z | 2 |
| V (Å³) | 2280.64 |
| D | 1.305 |
| R (%) | 5.07 |
| R_{w}(%) | 15.11 |
| G.o.F. | 1.045 |
| Reflections | 16242 |
| (Fₒ > 4sig(Fₒ)) | |
| Parameters refined | 594 |

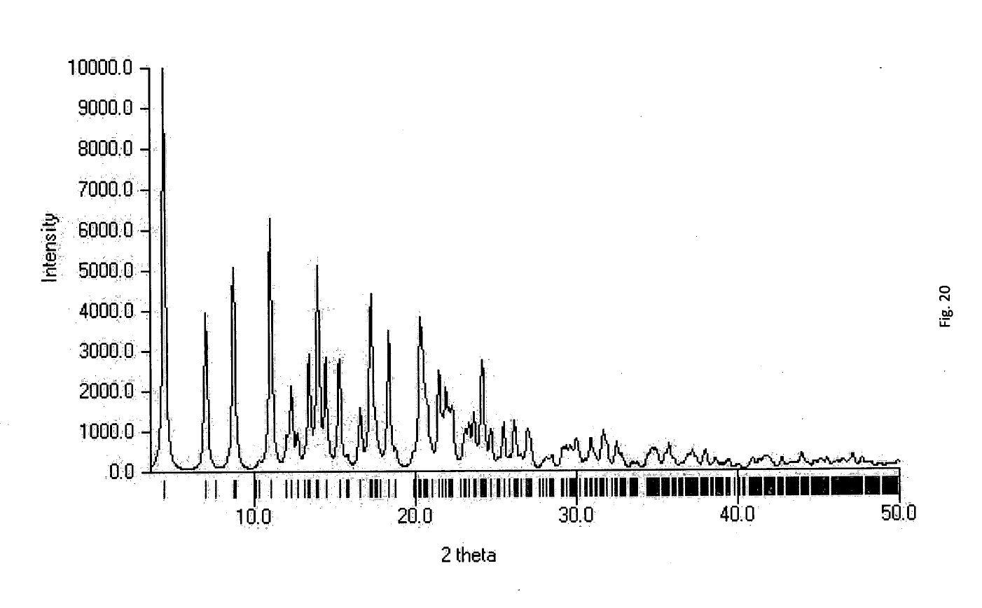

The experimental XRPD pattern of single crystal (FIG. 11) is analogous to the computer simulated pattern determined from single crystal X-ray diffraction data (FIG. 20). Based on X-ray studies of single crystal of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid, the hydrogen bonds in crystal were named. These bonds are between the following groups of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid: C═O(HCOOPr)—HO-7-HO-10 and C₉═O and also between groups of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate: C═O(Ac-4)-HO-1.

### Solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and Isopropyl Ester of Formic Acid

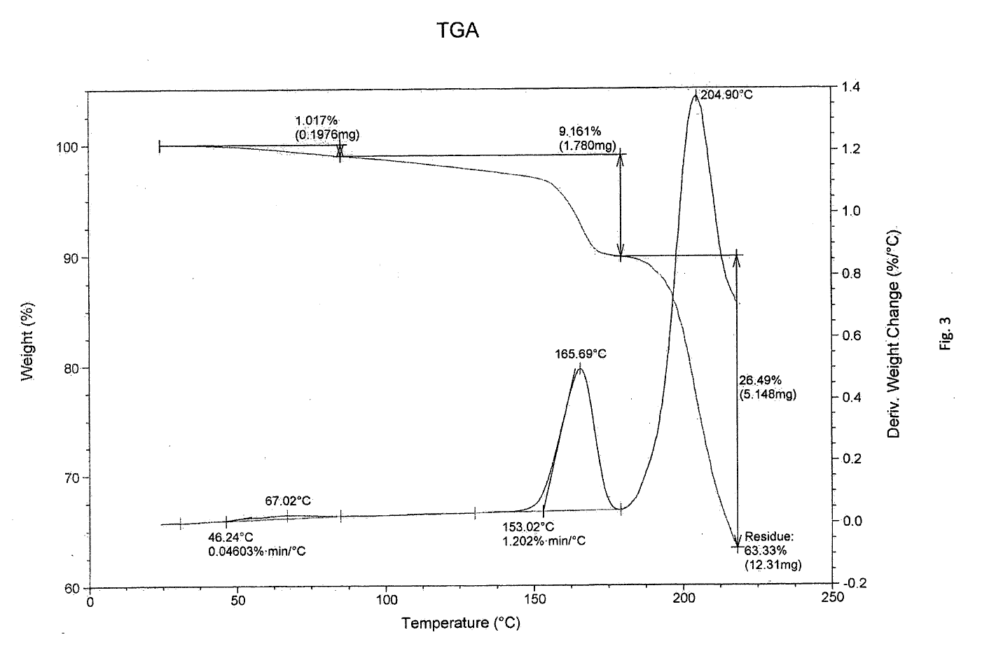

TGA curve (FIG. 3) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid shows 10.178% weight loss in the temperature range 30-180° C., especially in the range 45-175° C. After deduction of 0.181% of weight, which corresponds to free water determined by Karl-Fisher titration, the real weight loss corresponds to content of isopropyl ester of formic acid in solvate is 9.997% of initial sample weight. Theoretical content of isopropyl ester of formic acid in monosolvate is 9.834%.

¹H NMR of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid conforms stoichiometric composition of the solvate.

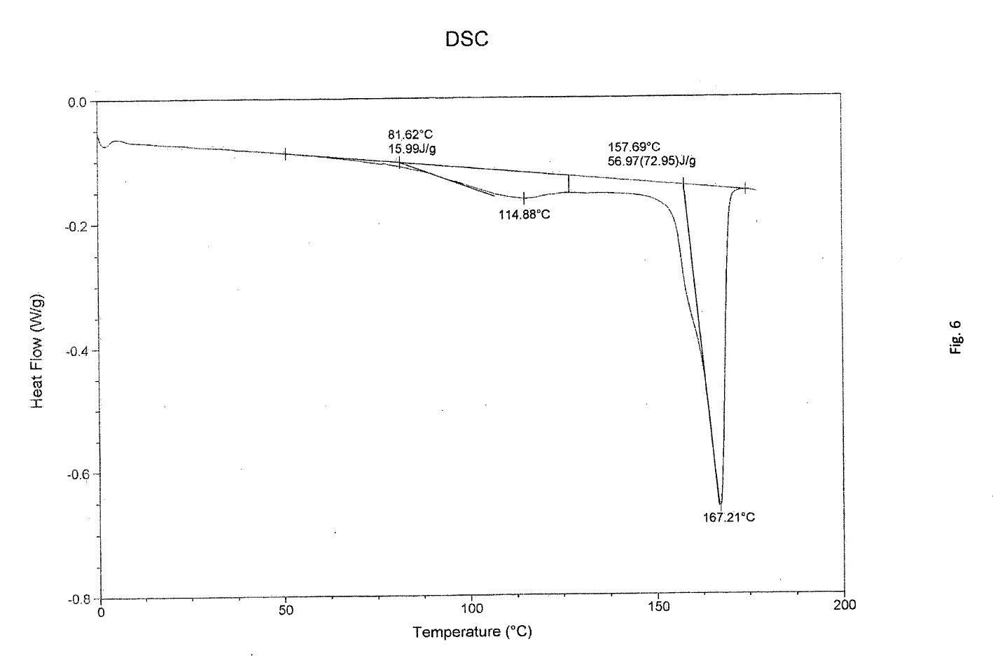

DSC (FIG. 6) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid shows no presence of hydration water.

FT-IR spectrum (Nujol) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid is characterized by bands at: 972, 1047, 1068, 1115, 1157, 1171, 1225, 1240 and 1265 cm⁻¹. The spectrum is characterized by additional bands at 1711, 3375, 3452 and 3491 cm⁻¹.

The solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid is characterized by X-ray powder diffraction (XRPD) pattern having peaks at 2θ values at: 4.6°; 7.2°; 9.0°; 11.2°; 14.1°; 15.4°; 17.4°; 18.5°; and 20.5±0.2°. The solvate can be characterized in detail by additional peaks at: 12.2°; 12.5°; 13.5°; 14.6°; 16.7°; 17.7°; 20.8°; 21.6°; 22.1°; 22.4°; 24.3°; and 26.3±0.2°.

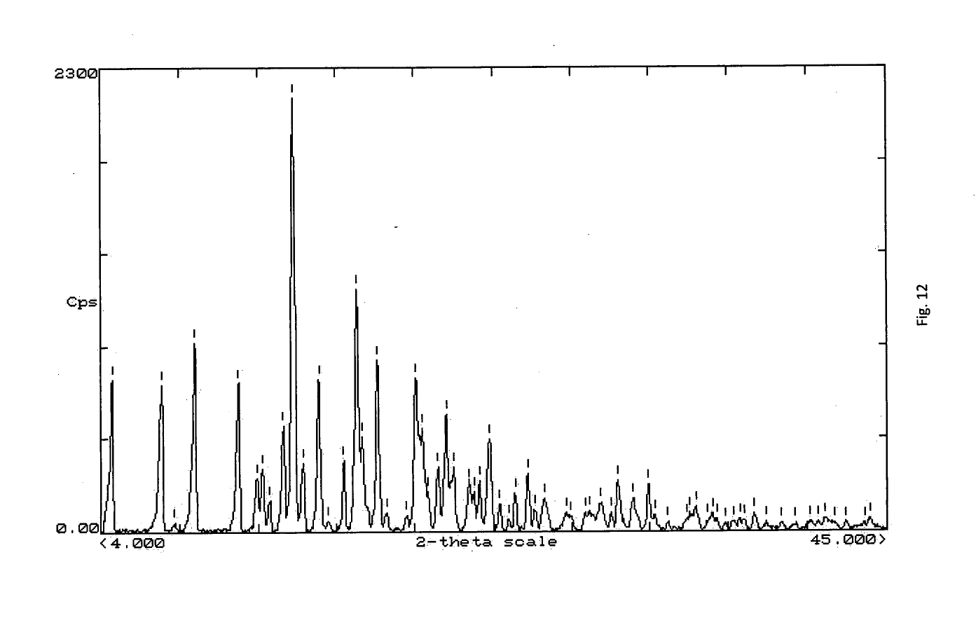

XRPD of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid obtained with CuKα₁ radiation source at wave length λ=1.54056 Å as the relation of relative intensity (I/Iₒ) and diffraction angles 2θ is substantially similar to the one shown in FIG. 12.

In Table 6 are listed measured 28 values, interplanar crystal spacing d values and relative intensities I/Iₒ, (intensities higher than 10%) in XRPD of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid:

TABLE 6
XRPD of solvate of 4-acetoxy-2α-benzoyloxy-5β,20-epoxy-1,7β,10β-
trihydroxy-9-oxo-tax-11-en-13α-yl (2R,3S)-3-tert-butoxycarbonylamino-2-
hydroxy-3-phenylpropionate and isopropyl ester of formic acid

| 2θ [°] | d [Å] | I/Iₒ [%] |
|---|---|---|
| 4.56 | 19.38 | 35 |
| 7.17 | 12.32 | 34 |
| 8.90 | 9.93 | 43 |
| 11.18 | 7.91 | 34 |
| 12.16 | 7.27 | 12 |
| 12.45 | 7.11 | 14 |
| 13.52 | 6.54 | 24 |
| 14.09 | 6.28 | 100 |
| 14.58 | 6.07 | 16 |
| 15.41 | 5.74 | 35 |
| 16.68 | 5.31 | 16 |
| 17.40 | 5.09 | 56 |
| 17.65 | 5.02 | 21 |
| 18.47 | 4.80 | 39 |
| 20.48 | 4.33 | 35 |
| 20.79 | 4.27 | 23 |
| 21.62 | 4.11 | 14 |
| 22.07 | 4.02 | 27 |
| 22.44 | 3.96 | 15 |
| 23.26 | 3.82 | 11 |
| 23.79 | 3.74 | 11 |
| 24.31 | 3.66 | 21 |
| 26.31 | 3.39 | 13 |
| 31.05 | 2.88 | 11 |
| 32.65 | 2.74 | 11 |

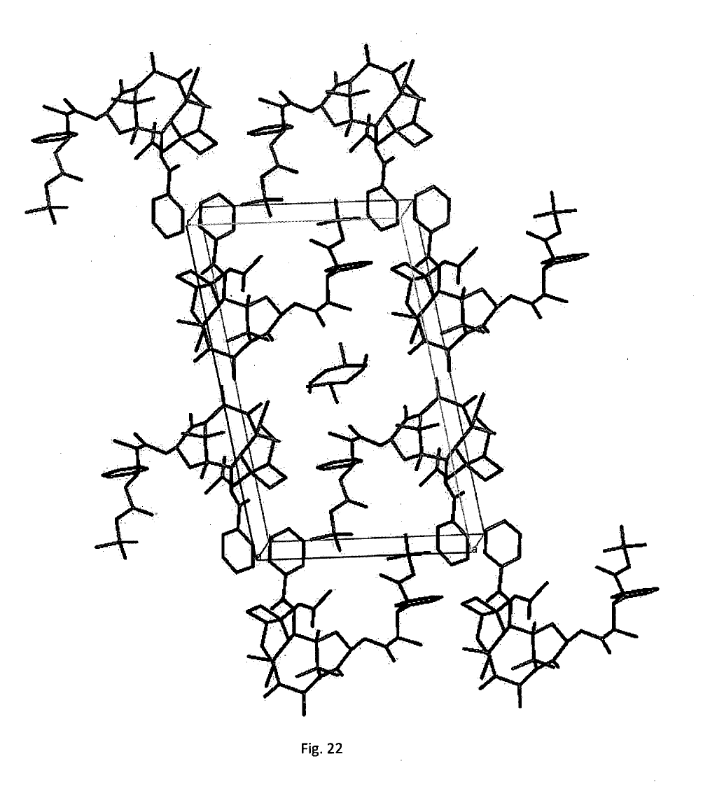

The crystalline structure (FIG. 22) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid was determined from single crystal X-ray diffraction data. The analysis conformed the presence of both 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid in the crystal lattice. Type of unit cell and parameter values of crystal lattice from single crystal X-ray diffraction studies conform that the crystal of solvate belongs to space group P2(1). Solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid crystallizes in monoclinic crystal system. Number of molecules in unit cell Z is equal to 2 considering calculated density d equal to 1.305. The full characteristic of crystalline form as list of parameters of unit cell is presented in Table 7.

TABLE 7
Parameters of unit cell of solvate of 4-acetoxy-2α-benzoyloxy-5β,20-
epoxy-1,7β,10β-trihydroxy-9-oxo-tax-11-en-13α-yl (2R,3S)-3-tert-
butoxycarbonylamino-2-hydroxy-3-phenylpropionate and isopropyl ester
of formic acid

| Crystal system | Monoclinic |
|---|---|
| Space group | P2(1) |
| a (Å) | 12.8515(5) |
| b (Å) | 8.7428(3) |
| c (Å) | 20.5416(8) |
| α (°) | 90 |
| β (°) | 99.004(1) |
| γ (°) | 90 |
| Z | 2 |
| V (Å³) | 2279.57 |
| D | 1.305 |
| R (%) | 3.48 |
| R_{w}(%) | 10.55 |
| G.o.F. | 1.031 |
| Reflections | 8728 |
| (Fₒ > 4sig(Fₒ)) | |
| Parameters refined | 596 |

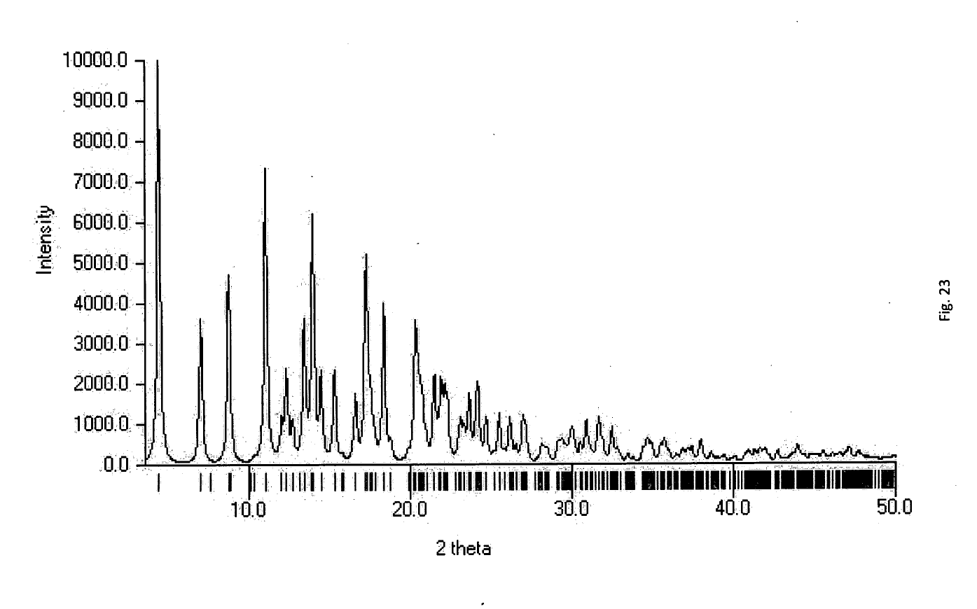

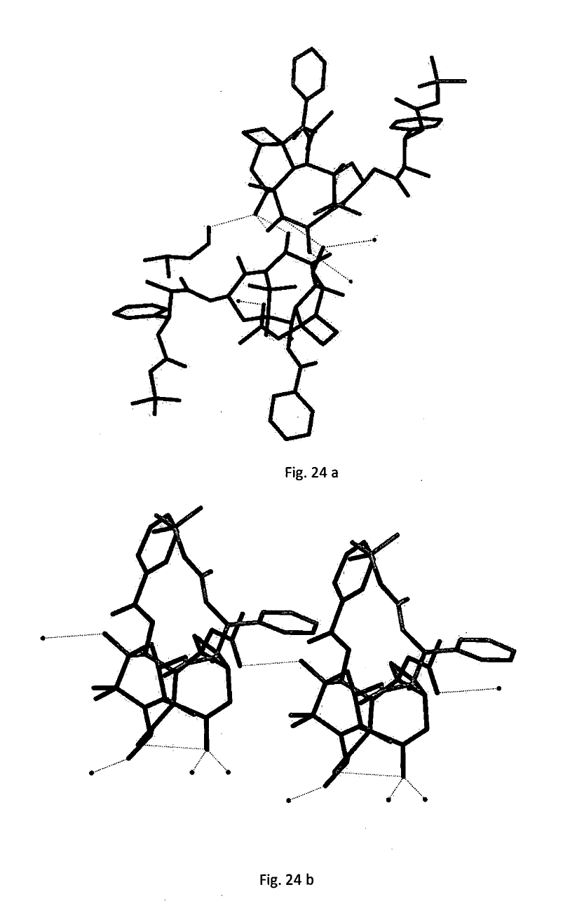

The experimental XRPD pattern of single monocrystal (FIG. 12) is analogous to the computer simulated pattern determined from single crystal X-ray diffraction data (FIG. 23). Based on X-ray studies of single crystal of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid, the hydrogen bonds in the crystal were named (FIG. 24). These bonds are formed between the following groups of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid: C═O (HCOOiPr)—HO-7-HO-10 and C₉═O and also between groups of 4-acetoxy-2α-benzoyloxy-5β,20-epoxy-1,7β,103-trihydroxy-9-oxo-tax-11-en-13α-yl (2R,3S)-3-tert-butoxycarbonylamino-2-hydroxy-3-phenylpropionate: C═O(Ac-4)-HO-1.

The crystalline solvates of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃-alkyl esters of formic acid obtained according to present invention are characterized by chromatographic purity (determined by HPLC method) higher than 99%.

The crystalline solvates of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃-alkyl esters of formic acid can be used, without any further purification, in the process of preparation of pharmaceutically pure 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate in the anhydrous or hydrate form, especially in the trihydrate form.

The process for the preparation of pharmaceutically pure 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate according to the invention comprises:
a) conversion of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate into its crystalline solvate with C₂₋₃ alkyl ester of formic acid,
b) desolvation of crystalline solvate obtained in step a),
c) isolation of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate in anhydrous or hydrate form, eg. preferably as trihydrate.

In the preferred embodiment of the invention, the process of desolvation in step b) may be carried out by crystallization of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate in the manner known per se, eg. from a solvent or the mixture of solvents, for example acetone and hexane as disclosed in U.S. Pat. No. 6,881,852, or ethanol and water as described in WO 96/01815, or acetonitrile and water as disclosed in U.S. Pat. No. 6,838,569. The crystallization results in obtaining 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate anhydrous or hydrate form, especially trihydrate form.

The invention is further illustrated by the following examples.

### EXAMPLES

### Analytical Methods

Thermal curves of differential scanning calorimetry were acquired using TA Instruments DSC2920 apparatus in aluminum crucible with sample heating rate equal to 5 K/m in.

Thermogravimetric measurements were made on TA Instruments TGA4950 apparatus in platinum pan with sample heating rate equal to 5 K/min.

Structural x-ray measurements were made on diffractometer Brucker Smart Apex 2 with MoKα radiation.

X-ray powder diffraction pattern were measured using X-ray powder diffractometer Seifert HZG-4 for CuKα1, λ=1.54056 Å radiation, range of 2θ from 4° to 45°, step size 0.04°, scanning rate 5 s at room temperature.

X-ray powder diffraction pattern measurements for the single crystal were made on PANanalytical X'Pert PRO MPD apparatus with radiation of CuKα at wave length λ=1.541874 in the 2θ range from 4° to 60° with step size 0.0167° and scanning rate 25 s at room temperature.

Obtained diffractograms were analyzed using EVA3.11 by Simens Analytical X-ray Instruments and X'pert Data Colector and X'pert Histore Plus both by PANanalytical software.

FT-IR spectra were registered in Nujol on Shimadzu FTIR-84005 spectrometer in the range of 4000-500 cm⁻¹ with 32 scans and resolution 4.0 cm⁻¹.

### Example 1

### Preparation of Solvate 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and Ethyl Ester of Formic Acid

9.673 g (11.25 mmol) of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate as trihydrate form were placed on Schott type funnel and were simultaneously dissolved in 210 mL of methylene chloride and filtered into flask. Solution was concentrated in continuous manner by means of evaporation on rotary evaporator. Water from trihydrate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate was azeotropic distillated off together with methylene chloride. Mixture was concentrated to 80 mL at bath temperature 38° C. Then evaporation of methylene chloride was continued with simultaneous addition of equal volume of ethyl ester of formic acid. 85 mL of ethyl ester of formic acid were added and crystallization mixture was concentrated to 60 mL. Then temperature of the mixture was gradually decreased to room temperature and the mixture was stirred at this temperature for one hour. Then crystallization mixture was placed in refrigerator ant left overnight at 0-3° C. Formed crystals were filtered on Schott funnel and washed twice with 12 mL of cold (about −15° C.) ethyl ester of formic acid. Crystals were dried on Schott funnel till reaching constant weight by stream of air of room temperature for one hour. Yield of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate was 9.006 g (10.22 mmol, 91%).

Structure of the solvate was confirmed by:

FT-IR (Nujol): 974, 1049, 1068, 1115, 1157, 1171, 1225, 1242 and 1267 cm⁻¹. The spectrum is characterized by additional bands at 1713, 3379, 3435 and 3501 cm⁻¹.

¹H NMR (500 MHz, DMSO-d₆, δ in ppm): 0.96 (s, 3H, H-16); 0.99 (s, 3H, H-17); 1.19 (t, 3H, HCOOCH₂CH₃); 1.34 (s, 9H, t-Bu); 1.50 (s, 3H, H-19); 1.61-1.69 (m, 2H, H-14 and H-6β); 1.72 (s, 3H, H-18); 1.84-1.89 (m, 1H, H-14); 2.20 (s, 3H, Ac-4); 2.22-2.28 (m, 1H, H-6α); 3.64 (d, 1H, H-3); 3.97-4.05 (m, 3H, H-7 and H-20); 4.12 (q, 2H, HCOOCH₂CH₃); 4.31 (t, 1H, H-2′); 4.51 (s, 1H, HO-10); 4.84-4.90 (m, 2H, H-5 and H-3′); 4.98 (d, 1H, HO-1); 5.03 (d, 1H, HO-7); 5.07 (s, 1H, H-10); 5.39 (d, 1H, H-2); 5.83 (t, 1H, H-13); 5.90 (d, 1H, HO-2′); 7.18 (t, 1H, Ph); 7.28 (d, 2H, Ph); 7.36 (t, 2H, Ph); 7.47 (d, 1H, NH), 7.61 (t, 2H, Bz), 7.70 (t, 1H, Bz), 7.95 (d, 2H, Bz), 8.18 (s, 1H, HCOOCH₂CH₃).

### Example 2

### Preparation of Solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and Propyl Ester of Formic Acid

479 mg (594 μmol) of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate were dissolved in 5 mL of methylene chloride. Solution was concentrated on rotary evaporator to about 2 mL at bath temperature 40° C. Then 4 mL of propyl ester of formic acid were added and evaporation of solvent was continued till reaching 3 mL of the remaining solution. Mixture was stirred on rotary evaporator till reaching room temperature. Then mixture was left without stirring at 2° C. for 12 hours. Crystals were filtered off on Schott filter funnel and washed with 1 mL of propyl ester of formic acid of temperature below 0° C. Yield after 1 hour of drying in stream of air of room temperature was 430 mg (489 μmol, 81%) of crystalline solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid.

FT-IR (Nujol): 974, 1049, 1068, 1115, 1157, 1171, 1225, 1242 and 1267 cm⁻¹. The spectrum is characterized by additional bands at 1713, 3383, 3439 and 3495 cm⁻¹.

¹H NMR (500 MHz, DMSO-d₆, δ in ppm): 0.88 (t, 3H, HCOOCH₂CH₂CH₃); 0.97 (s, 3H, H-16); 0.99 (s, 3H, H-17); 1.34 (s, 9H, t-Bu); 1.50 (s, 3H, H-19); 1.59 (sep, 2H, HCOOCH₂CH₂CH₃); 1.62-1.69 (m, 2H, H-14 and H-6β); 1.72 (s, 3H, H-18); 1.85-1.90 (m, 1H, H-14); 2.20 (s, 3H, Ac-4); 2.22-2.28 (m, 1H, H-6α); 3.4 (d, 1H, H-3); 3.97-4.05 (m, 3H, H-7, H-20 and HCOOCH₂CH₂CH₃); 4.31 (t, 1H, H-2′); 4.51 (s, 1H, HO-10); 4.84-4.90 (m, 2H, H-5 and H-3′); 4.96 (d, 1H, HO-1); 5.02 (d, 1H, HO-7); 5.08 (s, 1H, H-10); 5.39 (d, 1H, H-2); 5.84 (t, 1H, H-13); 5.88 (d, 1H, HO-2′); 7.18 (t, 1H, Ph); 7.28 (d, 2H, Ph); 7.36 (t, 2H, Ph); 7.44 (d, 1H, NH), 7.61 (t, 2H, Bz), 7.70 (t, 1H, Bz), 7.96 (d, 2H, Bz), 8.21 (s, 1H, HCOOCH₂CH₂CH₃).

### Example 3

### Preparation of Solvate of (2R,3S)-3-tert-butoxycarbonylamino-2-hydroxy-3-phenylpropionate 4-acetoxy-2α-benzoyloxy-5β,20-epoxy-1,7β,10β-trihydroxy-9-oxo-tax-11-en-13α-yl and Isopropyl Ester of Formic Acid

320 mg (397 μmol) of 4-acetoxy-2α-benzoyloxy-5α,20-epoxy-1,7α,10β-trihydroxy-9-oxo-tax-11-en-13α-yl (2R,3S)-3-tert-butoxycarbonylamino-2-hydroxy-3-phenylpropionate were dissolved in 3 mL of methylene chloride. Solution was concentrated on rotary evaporator to about 1 mL at bath temperature 40° C. Then 0.2 mL of isopropyl ester of formic acid were added and evaporation of solvent was continued till reaching 2 mL of the remaining solution. 1 mL of isopropyl ester of formic acid was added and evaporation of solvents was continued till reaching 2 mL of residual solution. Mixture was stirred on rotary evaporator till reaching room temperature. Then, during 4 hours, the temperature of not stirred mixture was decreased to about 2° C. Crystals were filtered off on Schott filter funnel. Yield after 1 hour of drying in stream of air of room temperature was 351.5 mg (393 μmol, 99%) of crystalline solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid.

FT-IR (Nujol): 972, 1047, 1068, 1115, 1157, 1171, 1225, 1240 and 1265 cm⁻¹. The spectrum is characterized by additional bands at 1711, 3375, 3452 and 3491 cm⁻¹.

¹H NMR (500 MHz, DMSO-d₆, δ in ppm): 0.97 (s, 3H, H-16); 0.99 (s, 3H, H-17); 1.20 (d, 6H, HCOOCH(CH₃)₂); 1.34 (s, 9H, t-Bu); 1.50 (s, 3H, H-19); 1.61-1.69 (m, 2H, H-14 and H-6β); 1.72 (s, 3H, H-18); 1.85-1.90 (m, 1H, H-14); 2.20 (s, 3H, Ac-4); 2.24-2.28 (m, 1H, H-6α); 3.64 (d, 1H, H-3); 3.98-4.06 (m, 3H, H-7 and H-20); 4.31 (t, 1H, H-2′); 4.51 (s, 1H, HO-10); 4.84-4.90 (m, 2H, H-5 and H-3′); 4.97 (m, 1H, HCOOCH(CH₃)₂); 4.98 (d, 1H, HO-1); 5.03 (d, 1H, HO-7); 5.08 (s, 1H, H-10); 5.39 (d, 1H, H-2); 5.84 (t, 1H, H-13); 5.90 (d, 1H, HO-2′); 7.18 (t, 1H, Ph); 7.28 (d, 2H, Ph); 7.36 (t, 2H, Ph); 7.47 (d, 1H, NH); 7.61 (t, 2H, Bz); 7.70 (t, 1H, Bz); 7.96 (d, 2H, Bz); 8.15 (s, 1H, HCOOCH(CH₃)₂).

### Example 4

### Growth of Single Crystals of Solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and Ethyl Ester of Formic Acid

1135 μmol of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate were dissolved in 10 mL of methylene chloride using rotary evaporator at bath temperature 38° C. Solution was concentrated by evaporation of about 3 mL. Solution was filtered through syringe type PTFE filter with 0.45 μm pores into crystallization dish of 120 mm of diameter. 45 mL of ethyl ester of formic acid warmed on bath of temperature 38° C. were added to the solution. Mixture was left overnight at room temperature for slow crystallization covering the dish partially with foil to allow slow evaporation of solvent. Solution was decanted after night and formed crystals were air dried. The yield of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid was 680 mg (772 μmol, 68%).

### Example 5

### Growth of Single Crystals of Solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and Propyl Ester of Formic Acid

568 μmols of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate were dissolved in 5 mL of methylene chloride and solution was concentrated by evaporation to volume of about 2.5 mL. 2.5 mL of methylene chloride were added and again solution was concentrated to volume of about 2.5 mL. Solution was transferred into crystallization dish of 90 mm of diameter and 22.5 mL of propyl ester of formic acid were added to the solution. Mixture was left for four days at room temperature for slow crystallization covering the dish partially with foil to allow slow evaporation of solvent. Solution was decanted after this time and formed crystals were air dried.

### Example 6

### Growth of Single Crystals of Solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and Isopropyl Ester of Formic Acid

159 μmols of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate were dissolved in 1 mL of methylene chloride and solution was concentrated by evaporation to volume of about 0.5 mL. 1 mL of methylene chloride were added and again solution was concentrated to volume of 1 mL. Solution was filtered through syringe type PTFE filter with 0.45 μm pores into crystallization dish of 40 mm diameter. 2.5 mL of isopropyl ester of formic acid were added to the solution. Mixture was left for four days at room temperature for slow crystallization covering the dish partially to allow slow evaporation of solvent. After four days solution was decanted and formed crystals were air dried.

### Example 7

### Purification of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate Via Solvate with Ethyl Ester of Formic Acid

From crude reaction mixture, after removal of protecting groups from 4-acetoxy-2α-benzoyloxy-5β,20-epoxy-1-hydroxy-9-oxo-7β,10β-bis(2,2,2-trichloroetoxy)carbonyloxy-tax-11-en-13α-yl (2R,3S)-3-tert-butoxycarbonylamino-2-hydroxy-3-phenylpropionate, containing product, zinc, tetrahydrofuran, acetic acid and other solid impurities, the product was isolated by the usual way, through: removal of solid impurities by filtration, concentration of the filtrate, extraction with methylene chloride and water, drying of organic extracts and concentration. Obtained crude product of HPLC purity of 93.91%, which contained not more than 0.83% for single known impurity and not more than 0.60% for single unknown impurity, was crystallized from ethanol-water in the manner described in WO 96/01815. Product of HPLC purity of 96.86% was obtained containing not more than 0.85% for single known impurity and not more than 0.23% for single unknown impurity (Waters SunFire 3.5 μm; 15×4.6 mm HPLC column analysis). Following the procedure described in Example 1, 500 mg (580 μmol) of described above product in the form of trihydrate were crystallized using methylene chloride and ethyl formate. 435 mg (493 μmol, 85%) of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid were obtained with HPLC purity of 99.18%. Product contained not more than 0.21% of known impurity (7-pT-DOC) and not more than 0.06% of unknown impurity (UnImp.RT41).

TABLE 9
HPLC purity of 4-acetoxy-2α-benzoyloxy-5β,20-epoxy-
1,7β,10β-trihydroxy-9-oxo-tax-11-en-13α-yl
(2R,3S)-3-tert-butoxycarbonylamino-2-hydroxy-3-phenylpropionate
prepared via solvate with ethyl ester of formic acid

| | Compound | | | | | | | | |
|---|---|---|---|---|---|---|---|---|---|
| Purity | 1 | 2 | 3 | 4 | 5 | 6 | 7 | 8 | 9 |
| Prior to | 0.02 | 0.01 | 96.86 | 0.24 | 0.22 | 0.01 | 0.23 | 0.74 | 0.85 |
| crystal- | | | | | | | | | |
| lization | | | | | | | | | |
| After | 0.00 | 0.01 | 99.18 | 0.11 | 0.17 | 0.01 | 0.06 | 0.12 | 0.21 |
| crystal- | | | | | | | | | |
| lization | | | | | | | | | |

### Wherein:

1 4-acetoxy-2α-benzoyloxy-5β,20-epoxy-1,7β,10β,13α-tetrahydroxy-9-oxo-tax-11-en (7-epi-DAB-III)
2 4-acetoxy-2α-[(1E)-1-methyl-prop-1-enoyl]oxy-5β,20-epoxy-1,7β,10β-trihydroxy-9-oxo-tax-11-en-13α-yl (2R,3S)-3-tert-butoxycarbonylamino-2-hydroxy-3-phenylpropionate (Imp. A)
3 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate (DOC)
4 4-acetoxy-2α-benzoyloxy-5β,20-epoxy-1,7β-dihydroxy-9,10-dioxo-tax-11-en-13α-yl (2R,3S)-3-tert-butoxycarbonylamino-2-hydroxy-3-phenylpropionate (10-oxo-DOC)
5 4-acetoxy-2α-benzoyloxy-5β,20-epoxy-1,7α,10β-trihydroxy-9-oxo-tax-11-en-13α-yl (2R,3S)-3-tert-butoxycarbonylamino-2-hydroxy-3-phenylpropionate (7-epi-DOC)
6 4-acetoxy-2α-benzoyloxy-5β,20-epoxy-1,7α-dihydroxy-9,10-dioxo-tax-11-en-13α-yl (2R,3S)-3-tert-butoxycarbonylamino-2-hydroxy-3-phenylpropionate (7-epi-10-oxo-DOC)
7 Unknown impurity RT 41 (UnImp. RT 41)
8 4-acetoxy-2α-benzoyloxy-5β,20-epoxy-1,7β-dihydroxy-9-oxo-10β-(2,2-dichloroethoxy)carbonyloxy-tax-11-en-13α-yl (2R,3S)-3-tert-butoxycarbonylamino-2-hydroxy-3-phenylpropionate (10-pT-DOC)
9 4-acetoxy-2α-benzoyloxy-5β,20-epoxy-1,10β-dihydroxy-9-oxo-7β-(2,2-dichloroethoxy)carbonyloxy-tax-11-en-13α-yl (2R,3S)-3-tert-butoxycarbonylamino-2-hydroxy-3-phenylpropionate (7-pT-DOC)

1. A solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃ alkyl ester of formic acid.

2. The solvate according to claim 1, which is a stoichiometric solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and ethyl ester of formic acid, comprising from about 8% to about 9 wt. % of ethyl ester of formic acid.

3. The solvate according to claim 2 characterized by X-ray powder diffraction pattern having peaks at 2θ values at: 4.5°; 7.1°; 8.8°; 11.1°; 14.1°; 15.4°; 17.4°; 18.5°; and 20.5±0.2°.

4. The solvate of claim 1, which is a stoichiometric solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and propyl ester of formic acid, comprising from about 9.4% to about 10.4% wt. of propyl ester of formic acid.

5. The solvate of claim 4, characterized by X-ray powder diffraction pattern having peaks at 2θ values at: 4.5°; 7.1°; 8.8°; 11.1°; 14.1°; 15.4°; 17.4°; 18.5°; and 20.4±0.2°.

6. The solvate of claim 1, which is a stoichiometric solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and isopropyl ester of formic acid, comprising form about 9.4% to about 10.4 wt. %. of isopropyl ester of formic acid.

7. The solvate of claim 6, characterized by X-ray powder diffraction pattern having peaks at 2θ values at: 4.6°; 7.2°; 9.0°; 11.2°; 14.1°; 15.4°; 17.4°; 18.5°; and 20.5±0.2°.

8. A process for the preparation of solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃ alkyl ester of formic acid, comprising crystallization of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate from a mixture of methylene chloride and C₂₋₃ alkyl ester of formic acid.

9. The process according to claim 8, comprising:
a) dissolving of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate, in either anhydrous or hydrate forms, in methylene chloride,
b) evaporation of methylene chloride,
c) addition of C₂₋₃ alkyl ester of formic acid,
d) optionally, seeding the crystallization mixture with crystals of the solvate,
e) leaving the crystallization mixture with, or without, stirring to form the crystals of the solvate,
f) isolation of the formed crystals,
g) washing of the crystals with C₂₋₃ alkyl ester of formic acid,
h) drying of crystals to constant weight.

10. The process according to claim 9, wherein steps b) and c) are carried out by simultaneous evaporating of methylene chloride to reach concentration of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate higher than 160 g/L while C₂₋₃ alkyl ester of formic is added.

11. The process according to claim 8, wherein the obtained solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃ alkyl ester of formic acid is of purity not less than 99% (by HPLC).

12. (canceled)

13. A process for the preparation of pharmaceutically pure 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate comprising:
a) conversion of the crude 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate in anhydrous or hydrate form, eg. trihydrate, into its crystalline solvate with C₂₋₃ alkyl ester of formic acid,
b) desolvation of the crystalline solvate obtained in step a),
c) isolation of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate in anhydrous or hydrate form, eg. trihydrate form.

14. The process according to claim 13, wherein desolvation in step b) is carried out by crystallization in which the solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃ alkyl ester of formic acid is put to the crystallization.

15. The process according to claim 13, wherein desolvation in step b) is carried out by crystallization from the solvent mixture water-ethanol or water-acetonitrile, and 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate is isolated in trihydrate form of purity higher than 99.0% (by HPLC).

16. The process according to claim 9, wherein the obtained solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃ alkyl ester of formic acid is of purity not less than 99% (by HPLC).

17. The process according to claim 10, wherein the obtained solvate of 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate and C₂₋₃ alkyl ester of formic acid is of purity not less than 99% (by HPLC).

18. The process according to claim 14, wherein desolvation in step b) is carried out by crystallization from the solvent mixture water-ethanol or water-acetonitrile, and 4-acetoxy-2α-benzoyloxy-5β,20-epoxy-1,7β,10β-trihydroxy-9-oxo-tax-11-en-13α-yl (2R,3S)-3-tert-butoxycarbonylamino-2-hydroxy-3-phenylpropionate is isolated in trihydrate form of purity higher than 99.0% (by HPLC).
